(19) United States
(12) Patent Application Publication
(10) Pub. No.: US 2023/0152296 A1
(43) Pub. Date: May 18, 2023

(54) METHOD FOR INCREASING THE QUALITY OF CRUDE OIL EXITING A GAS-OIL SEPARATION PLANT

(71) Applicant: SAUDI ARABIAN OIL COMPANY, Dhahran (SA)

(72) Inventors: Syed Abrar Ahmed, Dhahran (SA); Ashiff Khan Ka, Trissur (IN); Khaled Khalifah Al-Yousef, Dhahran (SA); Ahmad Hussain Al-Zubail, Dhahran (SA); Fayez Kharbat, Dhahran (SA); Amro K. Abbasi, Dammam (SA)

(73) Assignee: SAUDI ARABIAN OIL COMPANY, Dhahran (SA)

(21) Appl. No.: 18/051,788

(22) Filed: Nov. 1, 2022

Related U.S. Application Data
(60) Provisional application No. 63/278,864, filed on Nov. 12, 2021.

(51) Int. Cl. G01N 33/28

(52) U.S. Cl. CPC G01N 33/2847; G01N 33/2823

(57) ABSTRACT

A method for increasing the quality of crude oil exiting a gas-oil separation plant (GOSP) is disclosed. The GOSP hass sensors that determine process parameters of the crude oil, the method comprising determining, from the process parameters, WiO-parameters that depend on the concentration of water in the crude oil (WiO), determining virtual parameters of the crude oil, determining total parameters by adding the virtual parameters to the WiO-parameters, and performing a feedback loop. The feedback loop involves changing one or more of the total parameters, determining the quality of the crude oil exiting the GOSP, wherein when the quality is improved, the change in the one or more total parameters is maintained, when the quality is worsened, the change in the one or more total parameters is reversed, and repeating the feedback-loop as long as the quality of the crude oil exiting the GOSP increases.

### BACKGROUND

An oil field is a reservoir of crude oil in a formation. Operation of an oil field requires monitoring the quality of the crude oil regarding basic sediment and water (BS&W), salt, and oil in water which are residuals of unwanted impurities like water, sediment, and emulsions. The BS&W is the particulate matter suspended in the oil. An emulsion is a mixture of liquids that are unmixable. The BS&W is separated from the crude oil at the oil field to ease the transportation of the crude oil to a gas-oil separation plant (GOSP).

A GOSP separates the crude oil into vaporized gas and liquidated oil and water. To ensure the quality of the crude oil exiting the GOSP, the operators of the GOSP often rely on data sampling and in some cases on expensive online instruments or other methods that ignore the complex interplay of geometrical distribution of a multiphase fluid during flowing (flow regimes), chemicals, and emulsions comprising water and crude oil. As oil fields mature and water handling and disposal continues to increase in importance, quality predictions of the crude oil need to be improved for operators and engineers.

Accordingly, there exists a need for a method for increasing the quality of crude oil exiting a GOSP.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments disclosed herein relate to a method for increasing the quality of crude oil exiting a gas-oil separation plant (GOSP), wherein the GOSP comprises sensors that determine process parameters of the crude oil, the method comprising determining, from the process parameters, WiO-parameters that depend on the concentration of water in the crude oil (WiO), determining virtual parameters of the crude oil, determining total parameters by adding the virtual parameters to the WiO-parameters, and performing a feedback loop. The feedback loop involves changing one or more of the total parameters, determining the quality of the crude oil exiting the GOSP, wherein when the quality is improved, the change in the one or more total parameters is maintained, when the quality is worsened, the change in the one or more total parameters is reversed, and repeating the feedback-loop as long as the quality of the crude oil exiting the GOSP increases.

In general, in one aspect, embodiments disclosed herein relate to a non-transitory computer readable medium storing instructions executable by a computer processor, the instructions comprising functionality for determining, from the process parameters, WiO-parameters that depend on the concentration of water in the crude oil (WiO), determining virtual parameters of the crude oil, determining total parameters by adding the virtual parameters to the WiO-parameters, and performing a feedback loop. The feedback loop involves changing one or more of the total parameters, determining the quality of the crude oil exiting the GOSP, wherein when the quality is improved, the change in the one or more total parameters is maintained, when the quality is worsened, the change in the one or more total parameters is reversed; and repeating the feedback-loop as long as the quality of the crude oil exiting the GOSP increases.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency. The size and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn are not necessarily intended to convey any information regarding the actual shape of the particular elements and have been solely selected for ease of recognition in the drawing.

FIG. 1 shows a GOSP according to one or more embodiments.

FIG. 2 shows a cross-sectional view of the WOSEP according to FIG. 1, according to one or more embodiments.

FIG. 3 shows a flowchart of the method steps for increasing the quality of crude oil exiting a GOSP, according to one or more embodiments.

FIG. 4 shows a flow diagram of the method steps for increasing the quality of crude oil exiting a GOSP, according to one or more embodiments.

FIG. 5 shows the desalter of FIG. 1 according to one or more embodiments.

FIG. 6 shows a longitudinal cross-sectional view of a vessel of the GOSP, according to one or more embodiments.

FIG. 7 shows a transversal cross-sectional view of a vessel of the GOSP, according to one or more embodiments.

FIG. 8 shows a cross-sectional view of a coalescer, according to one or more embodiments.

FIG. 9 shows the dispersion of the flowing fluids as a function of the speed of the charge pumps, according to one or more embodiments.

FIG. 10 shows a GOSP similar to the GOSP of FIG. 1 that includes sensors that measure process parameters, according to one or more embodiments.

FIGS. 11A-11C show the impacts of the different components of the GOSP according to FIG. 1, according to one or more embodiments.

FIG. 12 shows a display with real-time output-values of the ML model and the impact of the process parameters on the quality of crude oil exiting a GOSP, according to one or more embodiments.

FIG. 13 shows the display of FIG. 12 with different output-values of the ML model.

FIG. 14 shows recommendations of the ML model, according to one or more embodiments.

FIG. 15 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

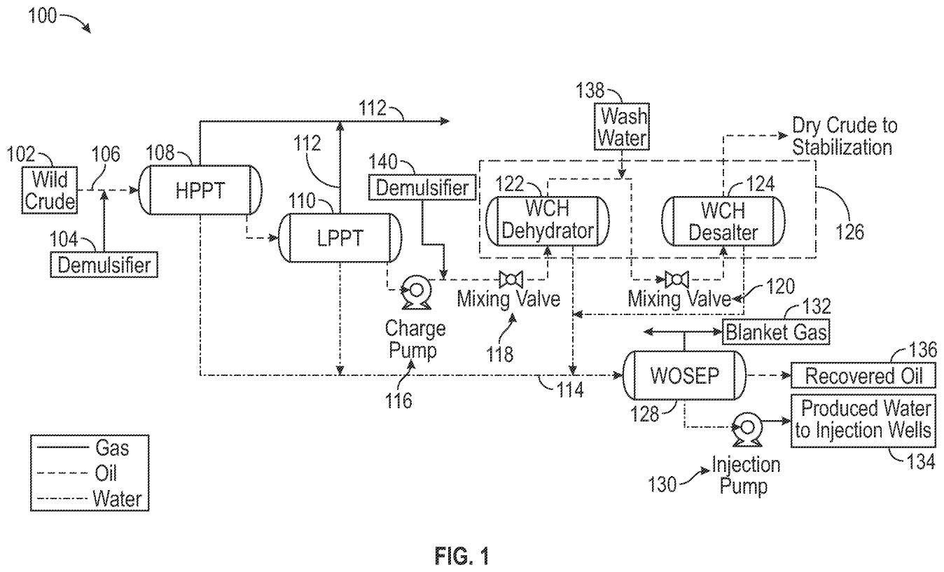

In the following description of FIGS. 1-13, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a seismic data set” includes reference to one or more of such seismic data set.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

In one aspect, embodiments disclosed herein relate to a method for increasing the quality of crude oil exiting a gas-oil separation plant (GOSP). The GOSP includes sensors that determine process parameters of the crude oil. The method involves determining WiO-parameters that depend on the concentration of water in the crude oil (WiO) from process parameters. Because the physical sensors that are present as part of the system have limitations on the measurements they are able to take, missing parameters are supplied by determining virtual parameters of the crude oil. Adding the virtual parameters to the WiO-parameters gives the total parameters required to train the system using machine learning. A feedback loop is employed to train the machine learning model with the parameters to predict the quality of crude oil exiting the GOSP and optimize the quality of the crude oil. In one or more embodiments, the feedback loop is repeated as long as the quality of the crude oil exiting the GOSP increases.

Embodiments of the present disclosure may provide at least one of the following advantages. The method for increasing the quality of crude oil exiting a GOSP provides a highly accurate way of increasing and monitoring the quality of crude oil and of analyzing the operator choices that impact the quality of the crude oil.

Furthermore, the method for increasing the quality of crude oil exiting a GOSP sustains product quality of the crude oil for BS&W and salt concentration and ensures that the product quality of the crude oil remains within the required targets of BS&W of 0.2% vol and salt concentration of 10 PTB. Therefore, the method helps plant operators to be one step ahead and to anticipate process upsets before they occur.

FIG. 1 shows a GOSP 100 with a dehydration stage and a desalting stage. The GOSP 100 includes a trunk line 106 that receives wild/wet crude oil 102 from an upstream manifold oil field. The crude oil from the oil field is a mixture of oil and water. The mixture is an emulsion that includes oil in water (OiW) as well as water in oil (WiO). The OiW is a dispersion of oil droplets in the water and the WiO emulsion is water droplets dispersed in the oil. In the trunk line 106, a first demulsifier 104 is added to the crude oil 102 that breaks emulsions in the crude oil 102. The crude oil is then fed into a three-phase separation vessel, i. e. High Pressure Production Trap (HPPT) 108. In the HPPT 108, the first stage of gas 112 and free water 114 is separated from the crude oil at a high pressure. The separated crude oil from the HPPT 108 is then fed into a second stage of the three-phase separation vessel, i.e., Low Pressure Production Trap (LPPT) 110 for further separation of more gas 112 and more water 114 from the crude oil at a lower pressure. In one or more embodiments, the crude oil from the LPPT 110 is also channeled through a wet/dry heat exchanger into a third stage Low Pressure Degassing Tank (LPDT) or separator that normally operates at almost atmospheric pressure (circa 3 psig) for more gas and water to be removed from the wet crude oil (not shown in FIG. 1).

The wet crude oil from the LPPT 110 or LPDT is transferred by crude oil charge pumps 116 into a Wet Crude oil Handling Facility (WCHF) 126 via mixing valves 118, 120. A second demulsifier 140 is added to the crude oil which is pumped by the crude oil charge pumps 116. In one or more embodiments, the first and second demulsifier 104, 140 are identical. In other embodiments, the first and second demulsifier 104, 140 are different. The WCHF 126 includes a Wet Crude Handling (WCH) dehydrator 122 and a single/double stage WCH desalter 124. Wash water 138 is added to the crude oil flowing from the WCH dehydrator 122 to the WCH desalter 124. In some embodiments, the wash water is water that is acidified with commodity acids, such as sulfuric, acetic, or citric acid. The type of acid is suited to the chemical profile of the crude oil. The pH-value of the wash water is between 5.5 and 7.0. The crude oil must be treated in the WCHF to meet the BS&W specification of 0.2 vol % and the salt concentration of less than 10 PTB.

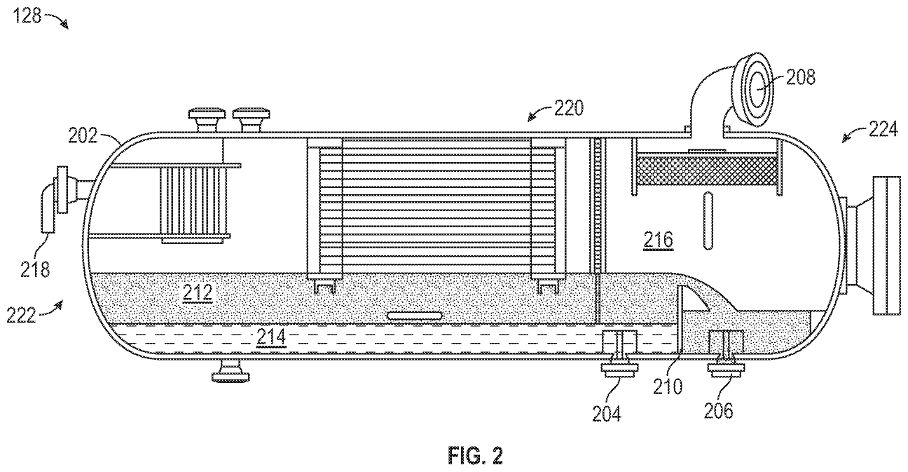

The dehydrated (dry) and desalted crude oil is then pumped into an atmospheric spheroid or degassing tank via a shipper pump and then flows to a crude oil stabilization column via a booster pump, or directly to a stabilizer (see stabilizer 202 in FIG. 2). At this point, the crude oil is stripped of volatile process parameters and stabilized to export grade crude oil specification of 13 True Vapor Pressure (TVP) at 130° F. The H₂S concentration is lowered to a required specification of 10 ppm wt. H₂S. In one or more embodiments, steam is injected into the crude oil after emerging from the reboiler and before entering into the crude oil stabilizer (see stabilizer 202 in FIG. 2). The produced export grade crude oil or stabilized oil is finally pumped by shipper pumps to an export terminal or refinery destination. The gas streams from the production traps 108, 110, degassing tank, and the stabilizer to the gas gathering compression system for onward delivery to the gas processing plants. Each stage of the compression plant consists of a gas compressor, a compressor discharge cooler, and a compressor discharge gas knock-out vessel.

The water stream from the production traps 108, 110, the WCH dehydrator 122, and the desalter 124 is pumped to a Water Oil Separator (WOSEP) 128. The WOSEP 128 recovers more oil (recovered oil 136) by separating the water. A gas (blanket gas 132) is maintained above the level of the crude oil in the tank of the WOSEP 128 to protect the crude oil inside the WOSEP 128 against air contamination. In some embodiments, the blanket gas is carbon dioxide, nitrogen, or any of the noble gasses, such as helium, neon, argon, krypton, or xenon. The blanket gas 132 also reduces the hazard of detonation of the WOSEP 128 and maintains normal operating pressure by pressurizing the crude oil in the WOSEP 128. Furthermore, the WOSEP 128 removes the oil concentration of inlet produced water to less than 100 ppm at the outlet as the disposal water, which is injected, via an injection pump 130, back into the reservoir (injection wells 134) for water-flooding and pressure maintenance.

The GOSP 100 further comprises sensors that measure the BS&W, salt, and WiO concentration in the crude oil exiting the GOSP. The measured BS&W, salt, and WiO concentrations are used, at least in part, to determine the measured quality of the crude oil exiting the GOSP.

FIG. 2 shows a cross-sectional view of the WOSEP according to FIG. 1. The WOSEP 128 is a horizontal three-phase WOSEP separating oil 212, water 214, and gas 216 from the crude oil. The WOSEP 128 comprises a cylindrical vessel 202 with an inlet 218 for entering the crude oil, a water outlet 204 for exiting the water 214, an oil outlet 206 for exiting the oil 212, and a gas outlet 208 for exiting the gas 216. Since gravity is the main separating force, the heaviest fluid (water) settles to the bottom of the vessel 202 and the lightest fluid (gas) rises to the top of the vessel 202. Therefore, the water outlet 204 and the oil outlet 206 are disposed on the bottom of the vessel 202 and the gas outlet 208 is disposed on the top of the vessel 202.

A weir plate 210 is used to block the heavier water and filter the lighter oil. The weir plate 210 is disposed on the bottom of the vessel 202 below the gas outlet 208 and between the water outlet 204 and the oil outlet 206. The vessel 202 is cylindrical and includes two base areas (heads) which are elliptically curved, wherein the inlet 218 is disposed on a first head 222 and the water outlet 204, the oil outlet 206, the gas outlet 208, and the weir plate 210 are disposed near a second head 224.

The degree of separation of the gas 216 and the water 214 depends on the operating pressure inside the vessel 202, the residence time of the fluids (e. g. oil, water, or gas) residing in the vessel 202, and the type of oil flow, because turbulent flow allows more bubbles to escape than laminar flow.

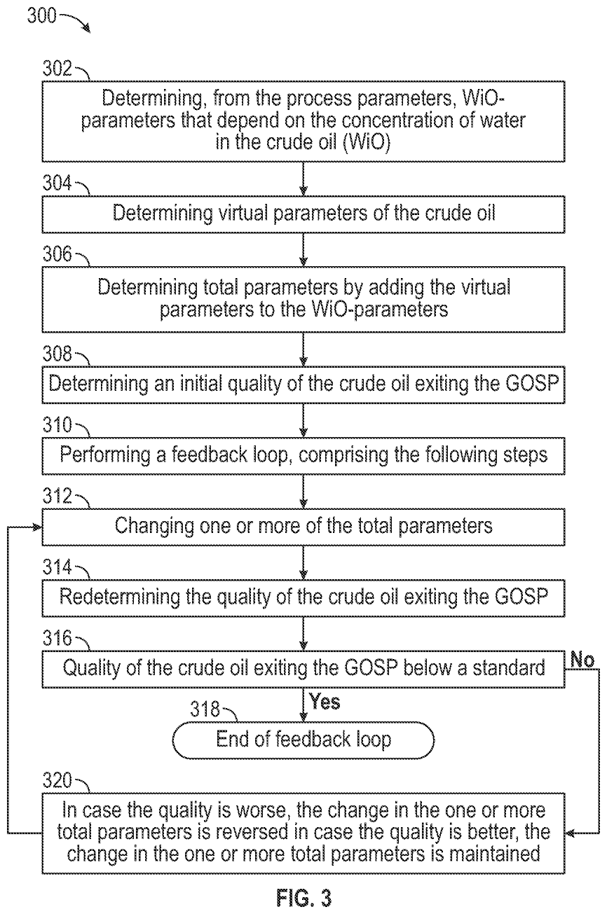

FIG. 3 shows a flowchart of the method steps for increasing the quality of the crude oil exiting the GOSP of FIG. 1.

In a first step 302, WiO-parameters that depend on the concentration of water in the crude oil (WiO) exiting the GOSP are determined from the process parameters.

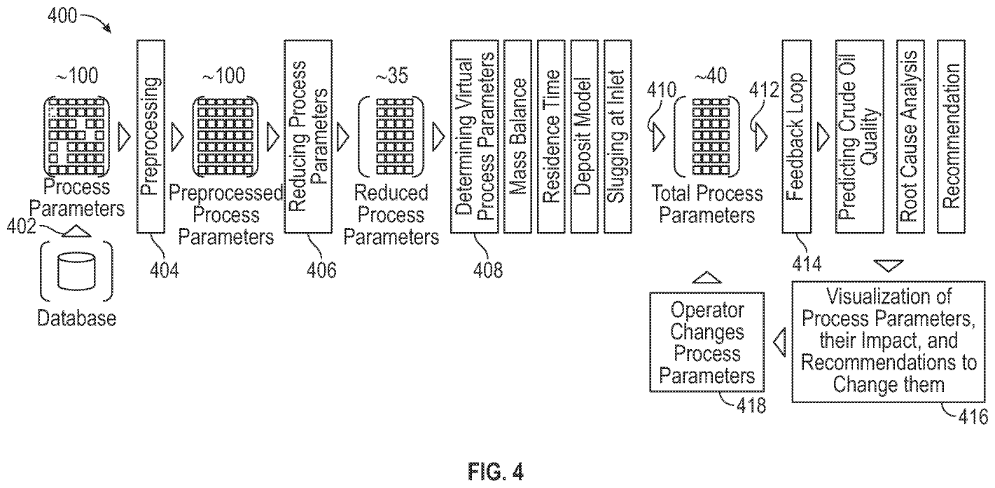

As discussed above, the GOSP includes sensors that determine the process parameters of the crude oil in the GOSP. In some embodiments, the process parameters include, but are not limited to, measurements such as flow rates in the pipes of the GOSP, salt concentration exiting the desalter, concentration of water exiting the LPPT, droplet size of water droplets in and out of the desalter, droplet size of water droplets in the HPPT or LPPT, droplet size in a coalescer, water concentration exiting the dehydrator, etc. These and other process parameters are explained in detail in step 402 of FIG. 4 below.

Some of the process parameters mentioned above depend on the WiO-concentration (WiO-parameters) in the crude oil exiting the GOSP and some of the process parameters do not depend on the WiO-concentration in the crude oil exiting the GOSP. A ML model is used to reduce the above process parameters to WiO-parameters. A more detailed explanation of this step is given in step 410 of FIG. 4.

In step 304, virtual parameters are determined. More specifically, the physical sensors of the GOSP present as part of the system may not measure all of the process parameters that depend on the WiO. The process parameters that are not able to be measured or observed by any of the physical sensors are missing parameters that are supplied by embodiments disclosed herein. These missing parameters are completed by virtual parameters obtained and predicted by the ML model. A more detailed explanation for this step is given in step 408 of FIG. 4.

In step 306, total parameters are determined by adding the virtual parameters to the WiO-parameters. For example, in case the number of the WiO-parameters is W and the number of the virtual parameters is V, the number of the total parameters is T=W+V. As the virtual parameters and the WiO-parameters depend on WiO, all the total parameters also depend on WiO as well. An example of determining the total parameters is given in step 410 of FIG. 4.

In step 308, an initial quality of the crude oil exiting the GOSP is determined. The quality includes the concentration of WiO, BS&W, and salt, in the crude oil exiting the GOSP. The lower the concentrations of WiO, BS&W and salt, the better the quality of the crude oil exiting the GOSP and vice versa. A more detailed explanation of this step is given in step 412 of FIG. 4.

In step 310, a feedback loop is performed (see step 414 of FIG. 4). The feedback loop includes steps 310-320 that are repeated as long as the quality of the crude oil exiting the GOSP is below a standard or predetermined threshold. Initially, the total parameters are entered into the feedback loop.

In step 312, one or more of the total parameters are changed. The changing of a total parameter is performed by the ML model. This is done to try and optimize the quality of the crude oil exiting the GOSP. In other words, the ML model is trained to try optimizing the total parameters by changing values of the parameters to try and obtain a high quality measure of the crude oil.

In step 314, the quality of the crude oil exiting the GOSP is redetermined. The redetermination of the quality of the crude oil exiting the GOSP is performed in the same manner as the determination of the quality of the crude oil exiting the GOSP according to step 412 of FIG. 4 with the changed total parameters.

In step 316, it is determined if the quality is below a standard. In one or more embodiments, the standard for the quality of the crude oil is a salt concentration in the crude oil less than 10 PTB and a BS&W less than 0.2%. In case the quality is below the standard, the feedback loop ends (see step 318). In case the quality is above the standard, the feedback loop continues with step 320.

In step 320, it is determined if the quality is better or worse. In case the quality is better, the change in the one or more total parameters is maintained. In case the quality is worse, the change in the one or more total parameters is reversed. A more detailed explanation of this step is given in sub-step “Recommendation” of step 414 of FIG. 4.

After step 320, the feedback-loop of steps 310-320 is repeated as long as the quality of the crude oil exiting the GOSP is above the standard. This step is explained in more detail in step 418 of FIG. 4.

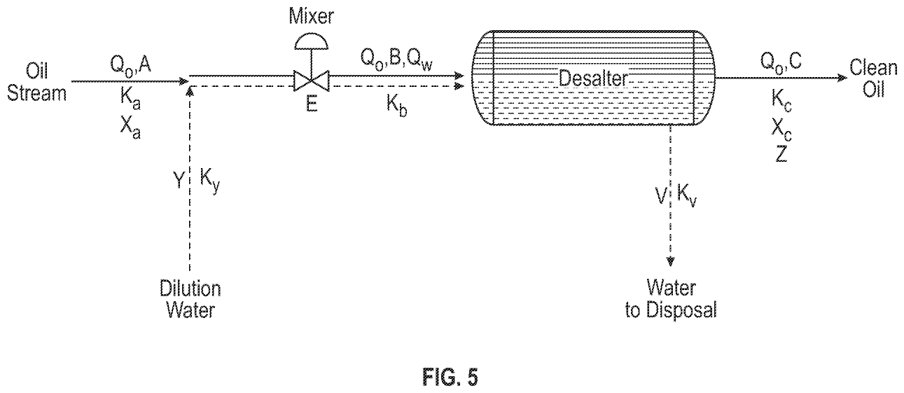

FIG. 4 shows an example flow diagram expanding on the method steps of FIG. 3 above, for increasing the quality of crude oil exiting a GOSP, according to one or more embodiments. Specifically, FIG. 4 expands on each of process parameters, virtual parameters, and the feedback loop with reference to FIGS. 5-9.

In a first step 402, process parameters of the GOSP saved in a database are gathered. In this example, the number of the process parameters is 100. Those skilled in the art will appreciate that the number of process parameters could be any suitable number, depending on the number of physical sensors that are part of the system, and the measurements obtained by these sensors.

As noted above, process parameters are, for example, the process parameters listed in step 302 of FIG. 3, measured by physical sensors present as part of the GOSP system. Each of the process parameters that may be measured are explained below.

1. Flow Rates in the Pipes of the GOSP

Some pipes of the GOSP in FIG. 1 include a sensor for the flow rate and some pipes don't include a sensor for the flow rate. The flow rate of the pipes without a flow rate sensor (missing flow rates) are calculated by the following mass Balance:

ΣQᵢₙₗₑₜ−ΣQₒᵤₜₗₑₜ=ΣΔV/t

where ΣQᵢₙₗₑₜ [m³/s] is the sum of all inlet flow rates, ΣQₒᵤₜₗₑₜ [m³/s] is the sum of all outlet flow rates, ΣΔV [m³] is the sum of all the changes in volume, and t [s] is the time between data points (granularity).

Changes in the volumes ΔV are calculated by ΔV=Vₜ₂−Vₜ₁, where V depends on the fill level and the dimensions of the vessel. The unknown flow rate is either part of the inlet flow rates ΣQᵢₙₗₑₜ or the outlet flow rates ΣQₒᵤₜₗₑₜ. To calculate the missing flow rate, the following equation may be used: Q_{?}=ΣQᵢₙₗₑₜ−ΣQₒᵤₜₗₑₜ, where the outlet flow rates are subtracted from inlet flow rates.

2. Salt Concentration Exiting the Desalter

FIG. 5 shows the desalter of FIG. 1. A mass balance of the desalter yields: Q₀+A+Y=Qₒ+C+V, where Qₒ [m³/s] is the flow rate of oil, A [m³/s] is the flow rate of the water from the dehydrator to desalter, Y [m³/s] is the flow rate of wash water into the desalter, C [m³/s] is the flow rate of the water out of desalter to stabilizer, and V [m³/s] is the flow rate of the water out to the disposal. This assumes that all oil from the dehydrator exits the oil outlet of the desalter.

Removing oil flow from both sides of the equation yields the water mass balance: A+Y=C+V. Efficiency through mixing valve yields: Y=A(K_{c}−Kₐ)/E(K_{y}−K_{c}), where Kₐ [kg/m³] is the salt content of water from the dehydrator to the desalter, K_{y} [kg/m³] is the salt content of wash water to desalter, K_{c} [kg/m³] is the salt content of water out of the desalter to the stabilizer, and K_{y} [kg/m³] is the salt content of the water out to the disposal.

Solving for K_{c} yields to K_{c}=(EYK_{y}+AKₐ)/(EY+A). The salt content in the clean oil is: mₛ=K_{c}X_{w}, where X_{w} is the measured water content downstream the desalter, and mₛ [kg/m³] is the salt mass per cubic meter oil.

The expected salt concentration may be calculated by the equation:

$$\frac{\begin{matrix}{CR}_{A} \times BS \& {W}_{DEH} \times {S}_{PW} \times 0.35051 + \\ \frac{MIXEFF}{100} \times {WWR}_{WW} \times {S}_{WW} \times 0.35051\end{matrix}}{{CR}_{A} \times BS \& {W}_{DEH} + \frac{MIXEFF}{100} \times {WWR}_{WW}} \times \frac{{CR}_{A} \times BS \& {W}_{DES}}{{CR}_{A}} ,$$

where CR_{A} (Crude Rate_{A}) is the crude oil production exiting the plant expressed in MBD (Thousand Barrels per day), S_{PW} (Salinity_{Produced Water}) is the salinity of the produced water determined from lab Geochemical analysis. Salinity is Total dissolved solids (tds) measured in water and expressed in mg/l, S_{WW} (Salinity_{WW}) is the salinity of the wash water determined from lab Geochemical analysis. Salinity is Total dissolved solids (tds) measured in water and expressed in mg/l, MIX EFF is Mixing Efficiency of the valve located upstream of the desalter and is epressed in percentage and is estimated from mixer valve opening, BS&W_{DEH} is the water in crude at outlet of dehydrator and is determined from sampling or online analyzers, BS&W_{DES} is the water in crude at outlet of desalter and is determined from sampling or online analyzers, and WWR_{WW}(Wash Water Rate_{WW}) is the rate of the water that is injected at inlet of TDS and is measured using flow meter.

The salt concentration in the crude oil exiting the desalter is measured in pounds of salt per thousand barrels of crude oil (PTB). Based on the known salt concentrations, the required flow rate of the wash water can be determined by the plant's personnel at all times.

The salt concentration in the crude oil is a process parameter that measures the quality of the crude oil. The salt concentration may be estimated using the WiO of the crude oil exiting the GOSP from real-time output values outputted by the ML model. Based on the calculated salt contents, the plants personnel will have the ability to identify the required wash water rate at all times.

3. Concentration of Water Exiting the LPPT

Assuming all water entering the dehydrator exits the dehydrator, it is possible to calculate the water entering the dehydrator from the LPPT by: [water volume from LPPT]=[volume change in dehydrator]+[volume water out of dehydrator]. Then the water concentration in the LPPT is: [water concentration out of LPPT]=[water volume from LPPT]/[total volume from LPPT], where [volume change in dehydrator] is based on water level in the dehydrator, [water volume out of dehydrator] and [total volume from LPPT] was calculated using the trapezoidal integral method. The flow rates are measured by sensors.

4. Droplet Size of Water Droplets in and Out of the Desalter (Two Characteristic Sized Droplets)

Assuming the droplets comprise two characteristic sizes: a small size from the water produced in the dehydrator, and a large size from the wash water. The dipole attraction force F_{c} in the direction of the electric field (θ=0) becomes:

$${F}_{c} = 24 π ϵ {E}^{2} \frac{{a}^{3} {b}^{3}}{{( {d}_{i} + a + b )}^{4}} .$$

Assuming the small droplets move much faster than the big droplets, an estimation of the coalescence time leads to:

$${T}_{R} = {T}_{c} = \frac{{d}_{i}}{{u}_{c}} .$$

Setting a coalescing force equal to the viscous drag force for the small droplets leads to:

$$6 π {μ}_{O} a \frac{{d}_{i}}{{T}_{R}} = 24 π ϵ {E}^{2} \frac{{a}^{3} {b}^{3}}{{( {d}_{i} + a + b )}^{4}} .$$

Since the small droplets move faster than the large droplets, the small droplets are used for the calculation of the viscous drag force F_{D}:

$$\frac{{a}^{2} {b}^{3}}{{{d}_{i} ( {d}_{i} + a + b )}^{4}} = \frac{{μ}_{o}}{4 ϵ {E}^{2} {T}_{R}} .$$

Assuming the size of the water droplets from the wash water are larger and represented by b, and further assuming dᵢ»b»a leads to:

$${a}^{2} = \frac{{μ}_{0} {d}_{i}^{5}}{4 ϵ {E}^{2} {b}^{3} {T}_{R}} , and$$

$$a = \sqrt{\frac{{μ}_{0} {d}_{i}^{5}}{4 ϵ {E}^{2} {b}^{3} {T}_{R}}} , and$$

$${d}_{R} = 2 a = \sqrt{\frac{{μ}_{0} {d}_{i}^{5}}{ϵ {E}^{2} {b}^{3} {T}_{R}}} .$$

Droplet Size for Large Droplet Radius

The droplets have a radius that makes the viscous drag force F_{D} of the droplets exiting the coalescer 800 with the oil stream equal to the force dragging the droplets down into the water. The droplet size is determined by the force balance:

w_{D}=F,

where w_{D} is the net weight (allowing for buoyancy) of a water droplet in the oil in [N], and F_{D} is the viscous drag force on a water droplet at speed uₒ of the oil flow in [m/s]. Inserting the force balance leads to:

$$\frac{π}{6} {b}^{3} ( {ρ}_{W} - {ρ}_{0} ) g = 3 π {μ}_{0} {u}_{o} b ,$$

where b is the diameter of the droplets in [m], uₒ is the velocity of oil flowing in [Pa], ρ_{w} is the density of the water in [kg/m³], ρₒ is the density of the oil in [kg/m³], and g is the gravity constant in [m/s²]. Estimating the velocity of the oil flowing inside the grid leads to:

$${u}_{O} = \frac{{Q}_{O}}{{A}_{grid}} .$$

Solving for b delivers:

$$b = \sqrt{\frac{18 {μ}_{O} {Q}_{O}}{{A}_{grid} ( {ρ}_{W} - {ρ}_{O} ) g}} .$$

Distance Between Droplets

Regarding the volume percentage of water in oil, it is assumed that all the droplets have equal size and are uniformly distributed. Assuming the average distance dᵢ (in [m]) between the water droplets is:

$${d}_{i} = {{d}_{P} ( \frac{\frac{4}{3} π}{X} )}^{\frac{1}{3}} ,$$

where X is the dispersed water volume fraction in [vol/vol], and dₚ is the average diameter of the droplets entering the vessel (for dehydrator the diameter calculated for LPPT is used) in [m]. A fixed value for dᵢ is used to calculate dₚ in the next component. It is further assumed that there are two sizes of droplets: a small size where the droplets are relatively close together, a large size where the droplets are relatively far apart. The average distance between the large droplets is estimated. It is further assumed, that the small droplets are uniformly distributed between the large droplets. An average maximum distance between a small droplet and a large droplet is half the distance dᵢ given above based on the large droplets alone. Averaging over a spherical volume leads to the distance between a small droplet and a large droplet of ⅜ of the distance dᵢ based on the large droplets alone.

5. Residence Time of Crude Oil in a Vessel of the GOSP

When crude oil enters a vessel of the GOSP, the crude oil remains in the vessel until the crude oil exits the vessel. The residence time T_{R} is the average time (in [s]) crude oil spends in a vessel. The residence time T_{R} is determined by dividing the volume V_{O} (in [m³]) of the crude oil in the vessel by the flow rate Q_{O} of the crude oil flowing in or out of the vessel in [m³/s]:

$${T}_{R} = \frac{{V}_{O}}{{Q}_{O}} .$$

The flow rate Q_{O} of the crude oil is either fetched from a flow rate sensor or calculated by the volume balance between the volume of the crude oil entering and exiting the vessel. The volume of the crude oil inside the vessel is calculated by the level of the crude oil in the vessel and the dimensions of the vessel.

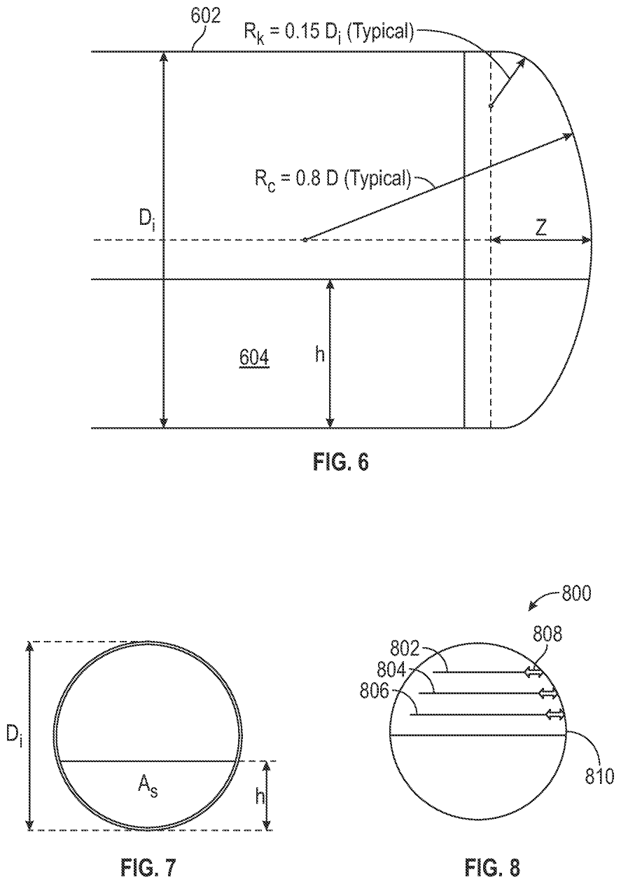

FIG. 6 shows a longitudinal cross-sectional view of a vessel of the GOSP. The vessel 602 has an inner diameter D (in [m]) and is filled with a crude oil 602, where h (in [m]) is the level of the crude oil in the vessel 602 measured from the bottom of the vessel 602. For example, the level h is the level of the water-oil interface. The vessel 602 has a 2:1 ratio according to the standards of the American Society of Mechanical Engineers (ASME 2:1).

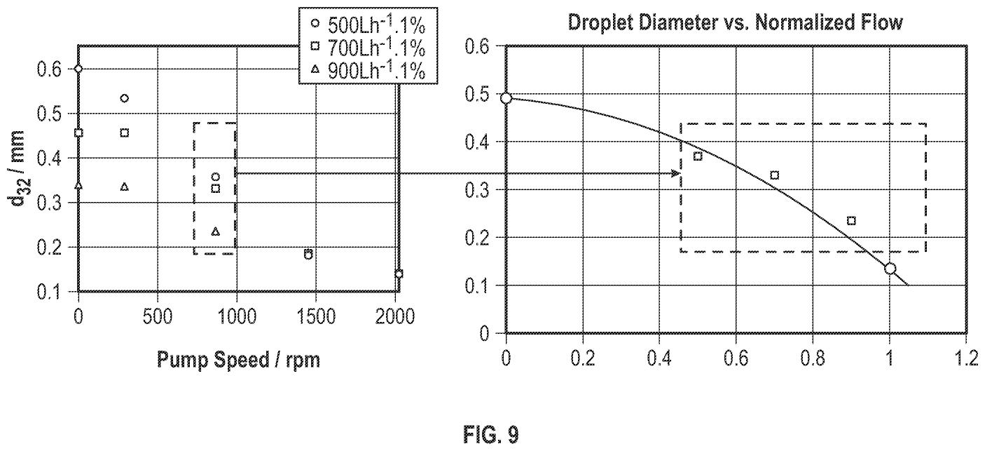

FIG. 7 shows a transversal cross-sectional view of a vessel of the GOSP. The volume V cylinder of the vessel is: V_{cylinder}=AₛL, where Aₛ is the cross-section of the vessel filled with liquid in [m²], and L is the length of vessel in [m].

The length L of the vessel 602 may be measured. The area Aₛ of the vessel filled with liquid depends on the level of the liquid inside the vessel 602. The cross-section of the vessel filled with the liquid is:

$${A}_{s} = \frac{( θ )}{4 * {D}^{2}} * \frac{1}{2} L ,$$

where θ is the central angle in the circular segment in [rad]. The angle θ is determined by:

$$θ = 2 ( 1 - 2 \frac{h}{D} ) .$$

The volume (in [m³]) of the vessel filled with liquid is:

$${V}_{cylinder - b} = L {D}^{2} ( \frac{1}{4} ( 1 - \frac{2 h}{D} ) - ( \frac{1}{2} - \frac{h}{D} ) \sqrt{\frac{h}{D} - {( \frac{h}{D} )}^{2}} ) .$$

In case of the vessel of the WOSEP, the volume V_{cylinder-b} is determined by the distance from the first head to the weir plate 210, and the level h measured by level sensors that are used for calculating the residence time.

In case the vessel is filled with water, which is heavier than crude oil, the volume V_{cylinder-b} of the vessel represents the volume of the water, with h as the level of the water-oil interface. In case the liquid is oil, the difference A_{O} between two cylindrical layers with different heights h is calculated by:

A_{O}=A_{S}(h_{GO})−A_{S}(h_{WO}),

where A_{S}(h_{GO}) is the cross-section of the vessel 402 from the height of the oil-gas interface to the bottom of the vessel 402 in [m²], and Aₛ(h_{WO}) is the cross-section of the vessel 402 from the height of the water-oil interface to the bottom of the vessel 402 in [m²]. Considering this, the volume V_{cylinder} of the vessel becomes: V_{cylinder-o}=L(Aₛ(h_{GO})−Aₛ(h_{WO})). The volume of the one of the elliptical heads is:

$${V}_{head} = \frac{{D}^{3}}{4} \frac{π}{6} ( 3 {( \frac{h}{D} )}^{2} - 2 {( \frac{h}{D} )}^{3} .$$

Similar as above, when the oil is floating between water (below the oil) and gas (above the oil), the volume of the oil in the head becomes:

V_{head-o}=V_{head}(h_{GO})−V_{head}(h_{WO})

6. Time Shift for Entering Process Parameters into the ML Model

The residence time is used to time shift the input of the process parameters to the ML model. The table below shows the time shift used for each of the different vessels:

| Vessel | Time shift |
|---|---|
| HPPT | 50 minutes |
| WOSEP | 50 minutes |
| LPPT | 30 minutes |
| Dehydrator | 10 minutes |
| Desalter | 0 minutes |

The method for increasing the quality of crude oil exiting a GOSP considers the residence time of the crude oil in each vessel of the GOSP, and is, therefore, a time-series method.

In the following example, the vessel of the WOSEP according to FIG. 2 is considered. Since there is less water than oil downstream of the weir plate 210, an oil droplet cannot settle in the water downstream of the weir plate 210. Hence, the length of the vessel for calculating an effective volume is measured from the first head with the inlet 618 to the weir plate 610. Therefore, the effective volume V_{eff} of the vessel for calculating the residence time is: V_{eff}=V_{cylinder}+V_{head}, where V_{cylinder} is the volume of the vessel in [m³], and V_{head} is the volume of the first head in [m³]. The total volume V_{O} of the vessel is: V_{O}=V_{cylinder}+2V_{head}.

7. Droplet Size of Water Droplets in the HPPT or LPPT

The largest water droplets escaping a vessel of the GOSP are the water droplets that do not descend into the water by gravity, but instead continuously float in the crude oil and exit the vessel with the oil stream. The size of the water droplets escaping the vessel depends on the separation efficiency. The higher the separation efficiency the smaller the size of the water droplets escaping the vessel. The maximum size of the water droplets that escape the vessel are the water droplets that have a residence time equal to the residence time of the oil in the vessel. This leads to the following equation:

T_{S}=T_{R},

where T_{S} is the residence time of the water droplets in the vessel in [s], and T_{R} is the residence time of the oil in the vessel in [s]. Estimating the residence time T_{S} of the water droplets leads to:

$${T}_{S} = \frac{{h}_{O}}{{u}_{D}} ,$$

where h_{O} is the height (thickness, depth) of the oil layer in the vessel in [m], and u_{D} is the velocity of the water droplets in the vessel in [m/s].

Each of the water droplets has a net weight w_{D} in the oil measured in [N]. Each of the water droplets also experiences a viscous drag force F_{D}, which depends on the speed u_{D} of the water droplet. A force balance of the water droplets leads to:

w_{D}=F_{D},

where w_{D} is the net weight of a water droplet in oil in [N], and F_{D} is the viscous drag force on a water droplet traveling at the speed u₀ of the oil flow in [m/s]. Thereby, the upward force exerted by the oil that opposes the weight of the water droplets (buoyancy) is considered. Consequently, the net weight w_{D} of the water droplet is:

$${w}_{D} = \frac{π}{6} {d}_{R}^{3} ( {ρ}_{W} - {ρ}_{O} ) g ,$$

where d_{R} is the average diameter of the water droplets in [m], ρ_{w} is the density of water in [kg/m³], ρₒ is the density of oil in [kg/m³], and g is the gravity in [m/s²]. The viscosity force on the water droplets moving through the oil is described by Stokes' law: f_{D}=3πμ_{O}u_{D}d_{R}, where μ_{O} is the (dynamic) viscosity of oil in [Pa·s]. The speed u_{D} of the water droplets is:

$${u}_{D} = \frac{{d}_{R}^{2} ( {ρ}_{W} - {ρ}_{O} ) g}{18 {μ}_{O}}$$

Assuming the roughly estimated balance T_{R}≈Tₛ leads to:

$${d}_{R} \approx \sqrt{\frac{18 {μ}_{O} {h}_{O}}{( {ρ}_{W} - {ρ}_{O} ) g {T}_{R}}} .$$

All water droplets with diameters bigger than d_{R} are likely to be separated in the vessel. For water droplets with a diameter smaller than d_{R}, only a fraction F(d) of the water droplets are separated. The fraction F(d) is estimated by the settling distance:

$$F ( d ) \approx \frac{h ( d )}{{h}_{O}} \approx \frac{{d}^{2}}{{d}_{R}^{2}} ,$$

where d is the size of the water droplets in [m] with d<d_{R}, F(d) is the fraction of water droplets with a diameter d<d_{R} that are separated in the vessel 402, and h(d) is the distance settled by water droplets of diameter d<d_{R} in [m].

The Reynolds number Re_{D} predicts the flow pattern of the water droplets in the oil. The flow of the water droplets in the oil is more laminar at low Reynolds numbers Re_{D}, while the flow of the water droplets in the oil is more turbulent at high Reynolds numbers Re_{D}. Stokes' Law only applies to water droplets with small Reynolds numbers Re_{D}:

$$R {e}_{D} = \frac{{ρ}_{O} {u}_{D} {d}_{R}}{{μ}_{O}} < 1 .$$

The table below shows calculated values for water droplets.

TABLE 1
Calculated values for water droplets.

| Property | Min | Max | Unit |
|---|---|---|---|
| Density | 800 | 850 | kg/m³ |
| Droplet velocity | 0.001 | 0.002 | m/s |
| Droplet size | 0.00005 | 0.0005 | m |
| Oil viscosity | 0.001 | 0.006 | Pa · s |

This yields a maximum Reynolds number of 0.85 when using only the (extreme) values above, indicating that Stokes' law is valid.

8. Droplet Size in Coalescer

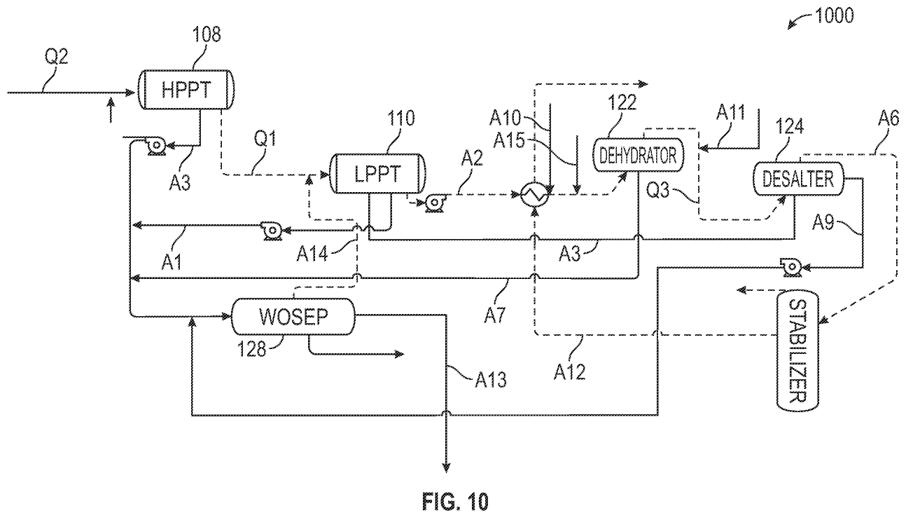

FIG. 8 shows a cross-sectional view of a dehydrator or a desalter, which is depicted a coalescer 800 that comprises a shell 810 and three electric grids: an upper grid 802, middle grid 804, and lower grid 806. The grids 802, 804, 806 comprise rods and make up the height of the coalescer 800 and have a clearance 808 to the shell 810 of the coalescer 800. The heights of the grids 802, 804, 806 are used, instead of level sensors and the clearance 810, to define a new diameter representing the circle around the grids 802, 804, 806. The coalescer 800 separates the oil-water emulsion into water and oil and merges the water droplets to a single water droplet and the oil droplets to a single oil droplet. The coalescer 800 is electrostatic and uses DC or AC electric fields or a combinations of DC and AC electric fields. The volume V of the oil in the electric grids 802, 804, 806 may be written as:

V_{g}=L(Aₛ(h_{upper-grid})−Aₛ(h_{lower-grid})),

where Aₛ is the cross-section of the cylinder at height h in [m²]. The equation for the cross-section of the cylinder is determined above. The diameter D_{g} (in [m]) of a cylinder where the rods of the electric grids 802, 804, 806 contact the shell 810 is: D_{g}=D−2t, where D is the inner diameter of the coalescer 800 in [m], t is the clearance 808 between a rod of the electric grids 802, 804, 806 and the shell 810 of the coalescer 800 in [m].

An estimation of the size of the droplets in a coalescer 800 is evaluated for representing the separation efficiency of the coalescer 800. However, the calculation of the size of the droplets in the coalescer 800 includes the electric field as an input to the equation, and since the electric field is affected by poor separation the calculated size of the droplets represents a consequence of poor separation instead of a cause.

Residence Time in Electric Coalescer

The residence time T_{Rg} inside the electric grids 802, 804, 806 of the coalescer 800 is the time the water droplets are subject to the electric field in the coalescer 800. The residence time T_{Rg} in the coalescer 800 is calculated similar to the residence time in the vessel 402, but the volume calculation depends less on the level sensors. The residence time T_{Rg} (in [s]) of the oil in the electric grids 802, 804, 806 of the coalescer is expressed as:

$${T}_{R g} = \frac{{V}_{g}}{{Q}_{o}} ,$$

where V_{g}=L(Aₛ(h_{upper-grid})−Aₛ(h_{lower-grid})) is the volume of the oil in the electric grids 802, 804, 806 in [m³] with Aₛ(h) [m²] is the cross section area of cylinder at height h, and Qₒ is the flow rate of oil out of the coalescer in [m³/s]. The equation for the cross sectional area of a cylinder is described above. The diameter used to calculate the cross section area for the electric grid is: D_{g}=D−2t, where D_{g} [m] is the diameter of a cylinder where the electric grid rods are touching the shell surface, D [m] is the inner diameter of coalescer, and t [m] is the clearance between rod and shell of coalescer.

Droplet Size Exiting Coalescer

The residence time T_{R} (in [s]) of the oil in the coalescer 800 is estimated by:

$${T}_{R} = \frac{{V}_{O}}{{Q}_{O}} ,$$

where V_{O} is the volume of the oil in the coalescer 800 in [m³], and Q_{O} is the flow rate of the oil into or out of the coalescer 800 in [m³/s]. The coalescence time (average time until two droplets coalescence) T_{c} (in [s]) of water droplets in the coalescer 800 is calculated by:

$${T}_{c} = \frac{{d}_{i}}{2 {u}_{c}} ,$$

where dᵢ is the average distance between the water droplets in [m], and u_{c} is the average velocity of the water droplets caused by dipole attraction in [m/s]. Since water molecules are dipoles, a dipole attraction force F_{c} (in [N]) attracts the water droplets to each other. In balance, the dipole attraction force F, is equal to the viscous drag force F_{D} (in [N]) on the water droplets moving at speed u_{c}: F_{c}=F_{D}. The viscous drag force F_{D} is estimated using the Stokes' Law (see above): F_{D}=3πμ_{O}u_{c}d_{R}, where μ_{O} is the (dynamic) viscosity of oil in [Pa·s], and d_{R} is the average size of the droplets in [m]. The dipole attractive force F_{c} (θ=0) in the direction of the electric field leads to:

$${F}_{c} = 24 πϵ {E}^{2} \frac{{b}^{3} {a}^{3}}{{( {d}_{i} + a + b )}^{4}} ,$$

where a is the radius of a droplet with a size a in [m], b is the radius of a droplet with the size b in [m], E is the dielectric constant in [C²/Nm²], E is the electric field in [V/m], and dᵢ is the distance between the closest surface of the droplets in [m]. Assuming all the droplets are of similar size leads to:

$$a = b = \frac{{d}_{R}}{2} , and {F}_{c} = \frac{3 πϵ {E}^{2} {d}_{R}^{6}}{8 {( {d}_{i} + {d}_{R} )}^{4}} .$$

Further assuming, that the attractive force F_{c} is equal to the viscous drag force F_{D} leads to:

$$\frac{3 πϵ {E}^{2} {d}_{R}^{6}}{8 {( {d}_{i} + {d}_{R} )}^{4}} = 3 π {μ}_{O} {u}_{c} {d}_{R} , and \frac{{d}_{R}^{5}}{{( {d}_{i} + {d}_{R} )}^{4}} = \frac{8 {μ}_{O} {u}_{C}}{ϵ {E}^{2}} .$$

Further assuming T_{R}≈T_{C} leads to

$${u}_{c} = \frac{{d}_{i}}{2 {T}_{R}} ,$$

and this leads to

$$\frac{{d}_{R}^{5}}{{{d}_{i} ( {d}_{i} + {d}_{R} )}^{4}} = \frac{4 {μ}_{O}}{ϵ {E}^{2} {T}_{R}} .$$

Further assuming dᵢ»d_{R} leads to:

$${d}_{R} = {{d}_{i} ( \frac{4 {μ}_{O}}{ϵ {E}^{2} {T}_{R}} )}^{1 / 5} ,$$

where d_{R} is the average diameter in [m] of the droplets exiting the coalescer 600.

9. Water Concentration Exiting the Dehydrator

A volume balance between the dehydrator and the desalter leads to:

Q_{i,dehydrator}+Q_{i,wash water}=Q_{o,crude}+Q_{o,disposal},

where Q_{i,dehydrator} is the flow rate of the crude oil out of the dehydrator in [m³/s] which is calculated from the total volume balance, Q_{i,wash water} is the flow rate of the wash water into the desalter in [m³/s], Q_{o,crude} is the flow rate of the clean crude oil out of the desalter in [m³/s], and Q_{o,disposal} is the flow rate of the water out to the disposal in [m³/s]. Assuming only water is added to the desalter, and the water is disposed through the water outlet, the following mass balance for the oil is obtained:

Q_{i,dehydrator}(1−X_{i,w})=Q_{o,crude}(1−X_{o,w}),

where X_{i,w} is the concentration of the water in the flow out of the dehydrator, X_{o,w} is the concentration of the water out of the desalter, which is measured by the analyzer. Solving for X_{i,w} leads to:

$${X}_{i , w} = \frac{( {Q}_{i , d e h y d r a t o r} - {Q}_{o , crude} ( 1 - {X}_{o , w} ) )}{{Q}_{i , dehydrator}} .$$

10. Dispersion Through Valves and Heat Exchanger

Dispersion of water waves means that waves of different wavelengths travel at different speeds. Water waves propagate on the surface with gravity and surface tension as the restoring forces. As a result, each water droplet disperses.

The Weber number analyzes fluid flows on the surface and is a measure of the relative importance of the fluid's inertia compared to its surface tension.

The valves, shell, and heat exchanger change the geometry of the flow compared to the pipelines. The change in geometry causes turbulent eddies and increases the dispersion of the water droplets. The Hinze-Kolmogorov equation in a simplified form is: dₘᵢₓ=kLWe−⅗, where dₘᵢₓ is the diameter of the water droplets after the mixing nozzle in [m], L is the large scale length (e. g. pipe diameter) in [m], We is the Weber number, and k is a constant which is quite small (˜0.06). The Weber number We is calculated by:

We=LΔp/σ,

where σ is the interfacial tension of the oil-water surface in [N/m], Δp is the pressure drop over a mixing valve in [Pa].

11. Pressure Drop Across Mixing Valves

The flow coefficient Cᵥ of a mixing valve measures the efficiency of the flow of the fluid through the mixing valve. The flow coefficient Cᵥ describes the relationship between the pressure drop across a mixing valve and the corresponding flow rate. The pressure drop through the mixing valves is estimated based on the flow rate and the flow coefficient Cᵥ curves supplied by the valve supplier. The flow coefficient Cᵥ (in [m³/(s Pa^{0.5})]) is calculated by: Cᵥ=Q\sqrt{SG/Δp}, where SG is the specific gravity of the fluid flowing through a mixing valve, and Δp is the pressure change across a mixing valve in [Pa]. Resolving for the pressure drop Δp leads to:

$$Δ p = S {G ( \frac{Q}{{C}_{v}} )}^{2}$$

The flow rate Q is known from rate measurements or mass balance and the flow coefficient Cᵥ depends on the flow rate Q: Cᵥ=f(Q). The relation between flow rate Q and flow coefficient Cᵥ is provided by the supplier of the valve. The valve is designed such that the flow coefficient follows a characteristic trend, and a table of flow rates with their respective flow coefficients is supplied.

12. Dispersion Across Charge Pumps

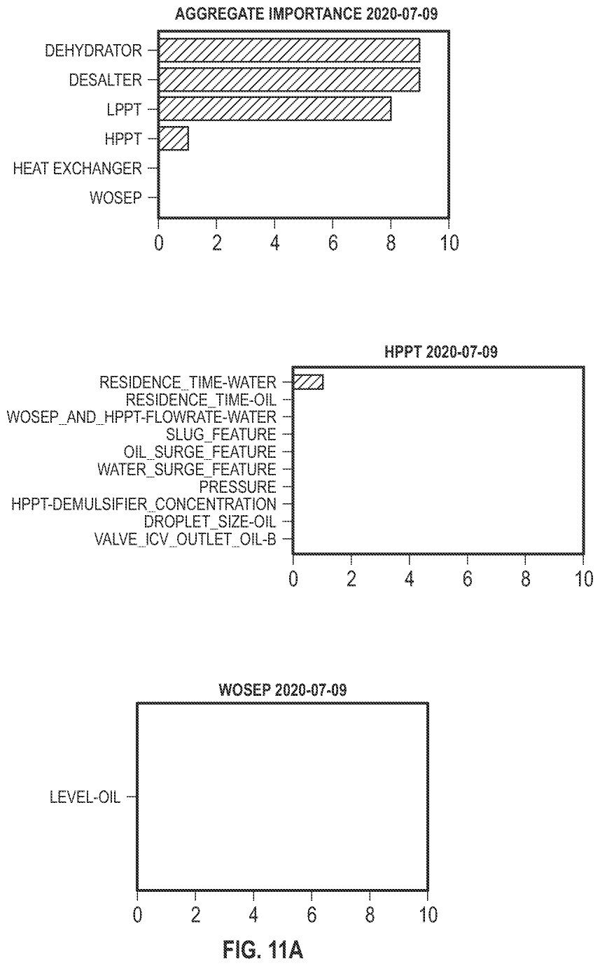

FIG. 9 shows the dispersion d₃₂ in [mm] of the flowing fluids as a function of the speed of the charge pumps. The fluids flowing through charge pumps will experience increasing dispersion with increasing pump speed and increasing flow rate. A centrifugal charge pump moves a fluid by transfer of rotational energy from driven rotors (impellers) that operate at a fixed pump speed such that only the flow rate is considered.

The flow rate through the charge pump is transformed to a relative size of the droplets by interpolating the values from the experiment using a quadratic function and adding two boundary conditions: 1. droplet size at 0 flow rate is larger than size of the droplets at the lowest flow rate (500 L/h), and 2. the flow rate at maximum flow rate is non-zero and lower than at the highest flow rate (900 L/h).

This technique transforms the flow rate through the pump, which has a non-linear relation to the separation efficiency, to a feature that has a more linear relation to the separation efficiency, as expressed by the droplet size. The calculated droplet size is a normalized droplet size and cannot be used in further calculations.

The constants for the second degree polynomial are a=−0.30249, b=−0.05324, and c=0.49 entered in the equation d=aQ²+bQ+c. To find the flow rate through each pump:

$$Q = \frac{{Q}_{t o t}}{{N}_{p u m p s}} ,$$

where Qₜₒₜ is the total flow rate through all the charge pumps in [m³/s], and Nₚᵤₘₚₛ is the number of active charge pumps. The number Nₚᵤₘₚₛ of the active charge pumps is found by looking at the number of open mixing valves into the charge pumps.

In step 404, the process parameters are preprocessed into preprocessed process parameters.

The ML model requires the values to be numeric and complete such that there are no gaps between the values. Therefore, the preprocessing includes: removing outliers (values more than certain standard deviations away from the mean), transforming binary values (numbers, characters, and graphic images) to binary values, e. g. whether a pump is on or off, transforming string values to numeric values, ensuring that the values have the same granularity in time, e.g., a value point every 1 minute, removing non-relevant values, and non-relevant periods are removed from the values so that they are not included in the training set, all missing values are replaced by linear interpolation, except binary values where a forward-fill is performed, and generating new set of calculated values to support the ML model from the collected values. Furthermore, all the missing process parameters are replaced by linear interpolation.

Moreover, bad values of the process parameters are removed. The bad values are identified by the following process. First, set point values of the process parameters are defined. A setpoint value represents the physical value of each process parameter. For example, the salt concentration in the oil exiting the GOSP should be less than 10 PTB. Thus, the set point value is 10 PTB for the process parameter salt concentration. The ML model identifies bad values, wherein a value is bad when the difference between the actual value and the set point values is unrealistically large.

In step 406, the preprocessed process parameters are reduced to reduced process parameters. In particular, the process parameters are reduced to the process parameters that have an impact on the quality, i. e., the WiO (WiO-parameter), of the crude oil exiting the GOSP. In this example, the number of the process parameters is reduced from 100 to 35. That means only 35 process parameters have an impact on the quality of the crude oil exiting the GOSP.

The ML model estimates WiO (target variable) in the crude oil exiting the GOSP, and the impact of each WiO-parameter on the WiO in the crude oil exiting the GOSP. When the WiO is high, the impact helps to understand which WiO-parameter contributed most to such a high WiO. Thus, the estimated WiO provides a measure of the accuracy of the ML model.

An estimated WiO close to the measured WiO indicates that the ML model has learned the behavior of the GOSP, such that the operator may trust the output of the ML model. Conversely, an estimated WiO deviating from the measured WiO indicates that the operator should practice caution when using results from the ML model.

The following table includes the process parameters of the GOSP according to FIG. 1 that have an impact on the WiO in the crude oil exiting the GOSP.

TABLE 2
List of process parameters of the GOSP that have an impact WiO in the crude oil exiting the GOSP.

| Component | Process Parameter |
|---|---|
| HPPT Separator | 1. oil droplet size |
| | 2. pressure |
| | 3. oil residence time |
| | 4. water residence time |
| | 5. concentration of the demulsifier |
| | 6. water flowrate in the WOSEP |
| | 7. oil outlet from valve lcv |
| LPPT Separator | 1. oil droplet size |
| | 2. oil residence time |
| | 3. oil flowrate in the dehydrator |
| | 4. pump charge dispersion flowrate |
| | 5. pressure |
| | 6. water flowrate in the WOSEP |
| WOSEP | 1. disposal water flowrate |
| Separator | 2. oil level |
| | 3. water level |
| Heat Exchanger | 1. degassed dispersion pressure difference |
| | 2. degassed temperature outlet |
| Dehydrator | 1. coalescer-dehydrator dispersion pressure diff mixing valve |
| | 2. coalescer-dehydrator WOSEP water flowrate |
| | 3. coalescer-dehydrator oil residence time |
| | 4. corrosion inhibitor drum dehydrator chemical flowrate |
| | 5. dehydrator-demulsifier concentration |
| | 6. coalescer-dehydrator water level |
| Desalter | 1. coalescer-desalter dispersion pressure diff mixing valve |
| | 2. coalescer-desalter stabilizer flowrate oil |
| | 3. coalescer-desalter temperature outlet oil |
| | 4. coalescer-desalter WOSEP flowrate water |
| | 5. drum-desalter wash water flowrate |
| | 6. coalescer-desalter oil residence time |
| | 7. coalescer-desalter water level |

In step 408, virtual parameters are determined. The following is a more detailed explanation of step 304 of FIG. 3.

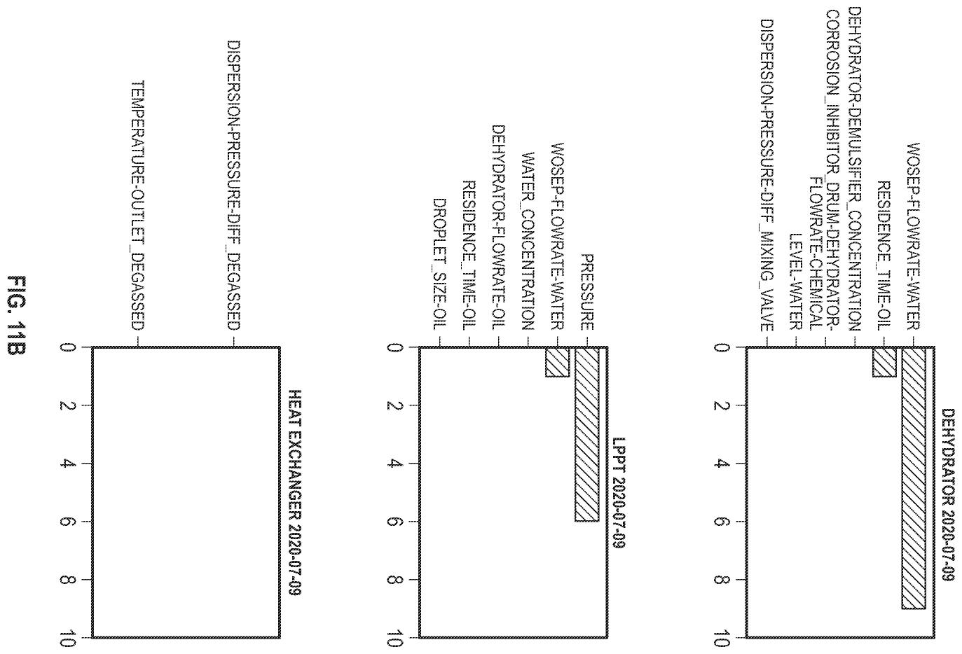

FIG. 10 shows a GOSP 800 similar to the GOSP 100 of FIG. 1 together with the flow rates Q₁, Q₂, Q₃, and A1-A15. For example, the crude oil flowing from the HPPT to the LPPT has the flow rate Q₁=A1+A2+ΔV/t, where ΔV/t=Vₜ₂−Vₜ₁, and V=f(levels, dimensions). The flow rate of crude oil flowing from the manifold oil field to the HPPT is Q₂=Q₁+A3+A2+ΔVₕₚₚₜ/t, where ΔVₕₚₚₜ/t=Vₜ₂−Vₜ₁, and V=f(levels, dimensions). The flowrate of crude oil flowing from the dehydrator to the desalter is Q₃=A6+ΔV_{desalter-oil}/t, where ΔV_{desalter-oil}/t=Vₜ₂−Vₜ₁, and V=f(levels, dimensions). V depends on the liquid levels controlled by the sensors in the vessels and on the dimension of the vessel. Table 3 below lists the flow rates Q₁, Q₂, Q₃, and A1-A15 shown in FIG. 10.

TABLE 3
Flow rates shown in FIG. 10

| Flow rates from | | |
|---|---|---|
| volume balance | Inputs | outputs |
| Q₁: Crude | A1(water from LPPT | A16 (recycled |
| oil from | to WOSEP) | crude oil |
| HPPT to LPPT | A2 (crude oil from LPPT to | from desalter) |
| | Dehydrator) | A4 (water from |
| | LPPT volume change | desalter) |
| Q₂ : Crude oil | Q₁ wet crude oil from | |
| from manifold | HPPT to LPPT | |
| to HPPT | A3 (water from HPPT to WOSEP) | |
| | HPPT volume | |
| Q₃ : Crude oil | A6 (crude oil from desalter to | |
| from dehydrator | stabilizer) | |
| to desalter | A7 (recycled water from desalter to | |
| | WOSEP) | |
| | Desalter oil volume ΔV_{desalter-oil}/t | |
| Desalter | A6 (crude oil from desalter to | A10 (demulsifier |
| | stabilizer) | to dehydrator) |
| | A9 (water outlet desalter WOSEP) | A11 (wash water |
| | Desalter water | to desalter) |
| | volume ΔV_{desalter-water}/t | |
| Stabilizer | A12 (crude oil from stabilizer to | |
| | wet dry crude heat exchanger) | |
| WOSEP | A13 Water from WOSEP to disposal | |
| | A14 Oil recycle from WOSEP to | |
| | LPPT | |
| Chemicals | A15 Corrosion inhibitor injection | |

As discussed above, some of the process parameters, such as some flow rates, that depend on WiO are missing. These missing process parameters are marked by a question mark “?” in FIG. 10. The missing process parameters, i. e., missing flowrates, need to be determined prior to the training of the ML model. Some flow rates of the GOSP 200 are listed in table 10 below.

TABLE 4
Flow rates from mass balance for outlet and inlet.

| Flow rate from mass balance | inlet | outlet |
|---|---|---|
| Crude oil from | A1 (LPPT to WOSEP, | A3 (recycled crude from |
| HPPT to LPPT | water) | desalter) |
| | A2 (LPPT to | A4 (water from desalter) |
| | Dehydrator, crude) | |
| | LPPT volume change | |
| Crude oil from | HPPT to LPPT wet | |
| Manifold to | crude | |
| HPPT | A5 (HPPT to WOSEP, | |
| | water) | |
| | HPPT volume | |
| Crude oil from | A6 (desalter to stabilizer | |
| Dehydrator to | crude) | |
| desalter | A7 (recycled crude from | |
| | desalter to LPPT) | |
| | Desalter oil volume | |
| Recycled water | A8 (desalter to LPPT) | A10 (demulsifier to |
| from desalter | A9 (water outlet desalter | desalter) |
| to desalter | to transfer pumps) | A11 (wash water to |
| | Desalter water volume | desalter) |

The missing process parameters are replaced with virtual process parameters. The ML model outputs the virtual parameters for the determination of the quality of the crude oil exiting the GOSP, and whether the ML model is accurately estimating the BS&W will be used to validate the results for the root cause analysis.

For example, the flow rate in the pipe going from the HPPT to the LPPT is calculated by the following considerations. The sum of the crude oil flowing into the LPPT is ΣQᵢₙₗₑₜ=Q_{?}+A3+A4. The sum of the crude oil flowing out of the LPPT is ΣQₒᵤₜₗₑₜ=A1+A2+ΔV/t. This yields: Q_{?}=A1+A2+ΔV/t−(A7+A4). According to the general equation above: ΣQᵢₙₗₑₜ=A2+A2+ΔV/t and ΣQₒᵤₜₗₑₜ=A3+A4.

Other examples for virtual parameters are slugging and surge.

Slugging

Slugging is accumulation of water, oil, or condensate in the pipelines of the GOSP. The ML model identifies slugging as a root cause whenever it is identified as important for calculating the BS&W in crude oil exiting the desalter.

In one or more embodiments, the flow of the crude oil in the pipelines of the GOSP is interrupted by liquid slugging. Slugs may be identified when there is an increase in the ratio of liquid flow rate to total fluid flow rate into the first separator, and this is used as slugging:

$${f}_{s} = \frac{{Q}_{l}}{{Q}_{l} + {Q}_{g}} ,$$

where Qₗ [m³/s] is the liquid flow rate, Q_{g} [m³/s] is the in situ gas flow rate, and fₛ is the slugging (ratio of liquid rate to total fluid rate). The gas rate is measured in standard conditions and is therefore converted to in situations using ideal gas law. With the pressure being 50 psi, it is assumed that ideal gas law is a good approximation here.

$${Q}_{g} = {Q}_{g s} \frac{{p}_{s}}{{T}_{s}} \frac{T}{p} ,$$

where Q_{gs} [m³/s] is the gas flow rate at standard conditions, pₛ [Pa] is the pressure at standard conditions (1 atm), p [Pa] is the pressure at in situ conditions in HPPT, Tₛ [K] is the temperature at standard conditions (60 F), and T [K] is the temperature at in situ conditions in HPPT. The liquid flow rate into the HPPT is calculated through mass balance.

The slugging is defined to identify a slug when the liquid to total fluid ratio is above 0.35. The number 0.35 is chosen based on reviewing historical data. For a slug incident the liquid to total fluid ratio is between 0.3 and 0.4. Therefore, the threshold is set to 0.35 which marks a conservative threshold as more severe slugs are expected to have even higher liquid to total fluid ratios.

Surge

Another virtual parameter that was developed as part of the ML model is the surge deployed in HPPT. A surge is the sudden rise or fall of pressure in a flowline. Surges are caused by the sudden closure of a valve.

During slugging, there are also surges in the oil level and water level that may have a large correlation with the increase of the BS&W. A linear model with a combination of oil and water level features across multiple time steps is created to prove that there is a strong correlation between oil- and water surges and rise of the BS&W.

Based on the strong correlation between surges and BS&W, a water and oil surge was developed. The intensity of the surges are captured by considering the standard deviation in the levels during an interval of 90 minutes. The interval length is based on the observation that the duration of the surges were typically more than 30 minutes and less than 90 minutes. To ensure that only true surges are captured there is a level threshold ensuring that startup periods are not identified as surges. The features included in the model are water surge: Standard deviation of water level in the HPPT over the past 90 minutes & water level >65%. When water level is below 65% the water surge is set to zero. Oil surge: Standard deviation (oil level in HPPT) over the past 90 minutes & oil level >70%. When oil level is below 70% the water surge is set to zero.

When reviewing results, with the slugging and surge added to the ML model, improvements are observed in the results of the ML model, especially when poor separation events are followed by surges and the ML model is associated with high impact on the surge.

In step 410, total parameters are determined by adding the virtual parameters to the WiO-parameters, according to step 308 of FIG. 3. In this example, five virtual parameters are added to the 35 process parameters to 40 total parameters.

In step 412, an initial quality of the crude oil exiting the GOSP is determined, according to step 308 of FIG. 3.

Machine Learning (ML) is deployed to predict the quality of crude oil exiting a GOSP. The predicted quality may be compared to the measured quality of the crude oil. For the prediction of the quality, the process parameters are entered into an ML model. In some embodiments, Intelligence (AI) is employed to understand the predictions of the ML model. The ML model reflects how each process parameter contributes to the prediction of the quality.

Furthermore, the ML model considers changes in the process parameters. This is done by finding an optimal training dataset for the current process parameters used to train the model. The ML model has access to all the historic process parameters. The historic input-values and the corresponding historic output-values are split randomly into training values and testing values such that the training values are 80% and the testing trained are 20% of the total values.

Whenever there is an increase of the quality in the crude oil exiting the GOSP, the ML model checks the impact of all process parameters on the quality of the crude oil exiting the GOSP for achieving this poor oil separation and highlights the sensors with high impact to the operator. The ML model predicts BS&W and salt in vol % of the oil.

In one or more embodiments, the ML model is based on multi-target time-series (hybrid AI). In this way, it is ensured that the quality of the crude oil exiting the GOSP is within the required targets of 0.2 vol % of the crude oil for BS&W and 10 pounds per thousand barrels (PTB) for the salt concentration. Therefore, management, engineers, plant operators are one step ahead of the GOSP and anticipate process upsets before they occur.

The ML model has two outputs. The first output is the predicted quality and the second output is the impact of the process parameters on the quality of the crude oil exiting the GOSP.

The first output, the predicted quality, provides a measure for the accuracy of the ML model. Whenever the predicted quality is close to the measured quality, it is an indication that the trained ML model has learned the behavior of the separation process, such that the operator can trust the output of the ML model. Conversely, when the predicted quality deviates from the measured quality, it is a sign that caution should be practiced by the operator when using results from the ML model and/or that the ML model needs further training

The second output, the impact of the process parameters, is a measure of how important a process parameter is for the quality of crude oil exiting the GOSP. Therefore, whenever the quality is high, the process parameters that contributed most to such a large quality are known.

The ML model has access to all the historic process parameters. Whenever there is an increase in the quality, the ML model looks through every process parameter to see which process parameters were important for achieving the increased quality and highlights this to the operator.

In one or more embodiments, the ML model uses a gradient boosting algorithm. The gradient boosting algorithm makes predictions in form of decision trees. When a decision tree is a weak learner, the algorithm of that decision tree is turned into a gradient-boosted tree to convert the weak learning decision tree into a strong learning decision tree. The decision tree algorithm is selected such that it handles multivariate problems where the target variable is non-linearly dependent on multiple time series, which is the case with water-oil separation, and has shown great results when applied to real world problems. The gradient boosting is performed by the software XGBOOST™, which identifies the root causes.

In other embodiments, the ML model uses scikit learn's gradient boosting regressor or random forest regressor instead of XGBOOST™. All three algorithms yield similar results. XGBOOST™ yields the best results combined with SHAP for finding local impacts.

In step 414, a feedback loop is performed, according to step 310 of FIG. 3. The feedback loop comprises the following steps 414-418 that are repeated as long as the quality of the crude oil exiting the GOSP is above a standard as defined in step 316 of FIG. 3.

Initially, the total parameters are entered into the feedback loop. Then, RCA is used for aggregating process parameters. For example, the WOSEP comprises three sensors and each sensor measures independently the level of the crude oil inside the WOSEP. Three sensors independently measuring the level of the crude oil inside the WOSEP has two negative consequences. The first negative consequence is that the impact of the level of the crude oil as a process parameter on the WiO in the crude oil exiting the GOSP is considered less important by the ML model. The second negative consequence is that duplicated input-values are entered to the ML model which slows the training of the ML model without improving the output of the ML model. This is a challenge for the RCA.

The steps taken for RCA are: Identifying sensors for the separation process, obtaining the process parameters that need to undergo the RCA, tailoring the ML model to identify a root cause when oil quality measurements contain too high water content, slug, or high salt.

A solution to this challenge is to sort all sensors and their process parameters and remove or aggregate redundant values. A method for aggregating a process parameter is averaging, maximizing, minimizing, and summing the values. Regarding the example above, values from two of the three sensors may be deleted or the three values of three sensors may be averaged (accounting for a third plant information sensor being out of calibration).

The table below shows an example of the aggregating features.

TABLE 5
Aggregating process parameters

| Aggregation Feature | Type |
|---|---|
| Average | Temperature and pressure |
| Maximum | Current |
| Minimum | Voltage |
| Average of closest 2 out of 3 | Level and pressure |

Sensors providing redundant process parameters may also be removed from the training set, which makes training the ML model faster. The improvement in the ML model is seen from a better accuracy of the ML model in terms of increasing WiO in crude oil when comparing with the actual WiO in the BS&W analyzer. This improvement in accuracy is a sign that the ML model has learned what is causing poor crude oil separation and thus can identify the root causes.

The root causes identified by the ML model are the most important factors contributing to the increased water concentration at a given time. Root causes are extracted from the ML model using Shapley values. Explainable AI (AI with understandable predictions, in contrast to black box AI with non-understandable predictions) is implemented in the open source SHAP Python library. SHAP calculates the importance for a set of process parameters X producing the output y when X is input to the ML model. This is true for any set of process parameters X, which means it is possible to extract local importance's, i. e. impact's at a specific point in time.

SHAP also takes the interactions between the process parameters into account, which is a key property for a root cause analysis tool dealing with varying conditions such as in a GOSP.

Next, the quality of the crude oil exiting the GOSP is redetermined. This step is performed according to step 412. In case the quality is below the standard, the feedback loop ends (see step 316 of FIG. 3).

Recommendation

In case the quality is above the standard, the feedback loop continues with making recommendations to increase the quality of the crude oil exiting the GOSP. The recommendations are directed to changes of the process parameters of the GOSP to improve the quality of the crude oil exiting the GOSP. The recommendations show the operator, which process parameters need to be changed in order to increase the quality of crude oil exiting the GOSP. The recommendations are directed to process parameters that have an effect on the quality.

The recommendations are advisory recommendations to an operator of the GOSP. The advisory recommendations can be explained as follows.

The ML Model finds optimal process parameters. The ML model estimates BS&W of the crude oil exiting the GOSP starting with an initial process parameters. By changing the process parameters and observing how the quality of the crude oil exiting the GOSP changes, the ML model optimizes the process parameters to obtain the highest possible quality of the crude oil exiting the GOSP.

A bounded optimizer is used to optimize the process parameters. All process parameters are considered individually by finding an optimal value for one process parameter while keeping all other process parameters at a fixed value. The optimal value for the one process parameter is the value that causes the model to estimate the lowest possible BS&W in the crude oil exiting the GOSP.

For each of the process parameters a lower and upper bound is used to search for the minimum BS&W estimated by the ML model. The bounds used are the 10th and 90th percentile values for the process parameter available in the training dataset. This ensures that only values previously acquired by the ML model are used for optimization.

When optimal values for all process parameters have been identified they are ranked based on which process parameters reduce BS&W the most. In addition, whether a process parameter is too high or too low is part of the output and is found by comparing the current setpoints of the process parameters with the optimal values. The top five process parameters in the ranking are made available to the end users. This is to focus attention only to the top five most important process parameters for the given situation. The top five most important parameters are highlighted.

These process parameters are marked with underlining in the table below. The remaining process parameters are used to calculate features going into the ML model, and then these calculated features have been input to the optimization scheme. Since these features are either negatively or positively correlated with the process parameter their correlation is used to determine the direction to change the process parameter. Some process parameters are correlated with more than one calculated feature. In this example, the feature with the highest impact on BS&W is used.

TABLE 6
Process parameters with correlated features. Process parameters that are input to the ML model are underlined.

| Process | Positively | Negatively |
|---|---|---|
| parameter | correlated feature | correlated feature |
| HPPT oil level | HPPT Oil residence time | HPPT Maximum |
| | | Water droplet size |
| | | escaping separator |
| HPPT water level | HPPT Water residence time | HPPT Water outlet |
| | HPPT Oil outlet | rate |
| | valve opening | |
| LPPT oil level | Oil outlet rate | LPPT oil residence time |
| LPPT water | LPPT water outlet rate | LPPT Water |
| outlet rate | | concentration in oil |
| | | outlet to dehydrator |
| WOSEP oil level | WOSEP oil level | |
| Heat exchanger | Heat exchanger Temperature | |
| Temperature outlet | outlet of degassed fluid | |
| of degassed fluid | | |
| Dehydrator water | Dehydrator water level | Dehydrator water |
| level | | outlet rate |
| Desalter water | Desalter water level | Desalter water |
| level | | outlet rate |
| Desalter oil outlet | Desalter oil outlet | |
| Demulsifier rate to | Dehydrator demulsifier | |
| dehydrator | concentration | |
| Demulsifier rate to | HPPT demulsifier | |
| HPPT | concentration | |
| Wash water rate | Wash water rate | |

In step 416, the process parameters, their impact, and recommendations to change them are visualized to an operator.

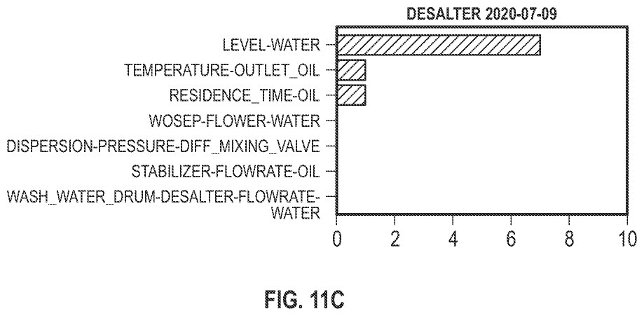

FIG. 11 shows the plots of various components of the GOSP and their process parameters and their impact on the quality of the crude oil exiting the GOSP. The plots are outputted by the ML model. The impact is plotted on a relative scale from 0 to 10.

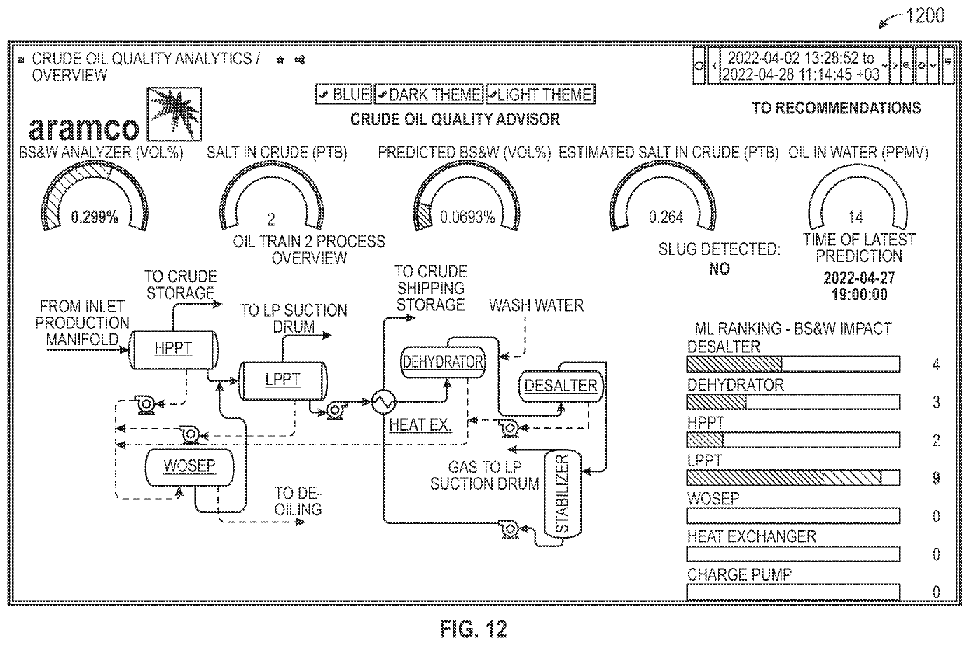

FIG. 12 shows a display with real-time output-values of the ML model and the impact of the process parameters on the quality of crude oil exiting a GOSP, according to one or more embodiments.

The display 1200 shows the BS&W in the oil exiting the GOSP at 0.299 vol %, the actual salt concentration in the oil exiting the GOSP at 2 PTB, the predicted concentration of BS&W in the oil exiting the GOSP at 0.063 vol %, and the estimated concentration of salt in the crude oil exiting the GOSP at 0.264 PTB.

The display 1200 shows, on a lower right part, the components of the GOSP that have an impact on the BS&W and salt concentration in the oil exiting the GOSP. For example, the LPPT has an impact of 9 and therefore the highest impact on the BS&W and salt concentration in the oil exiting the GOSP. The heat exchanger has an impact of 0 and therefore no impact on the BS&W and salt concentration in the oil exiting the GOSP.

The BS&W in the oil exiting the GOSP at 0.299 vol % is higher than the standard of BS&W being less than 0.2 vol %. Since the LPPT has the highest impact on the BS&W, the ML recommends changing process parameters of the LPPT that have an impact on the BS&W and repeating the feedback loop until the BS&W is less than 0.2 vol %.

In one or more embodiments, the real-time output-values of the ML model comprise BS&W, salt concentration, and water concentration of the crude oil exiting a GOSP and is, therefore, a multi-target method.

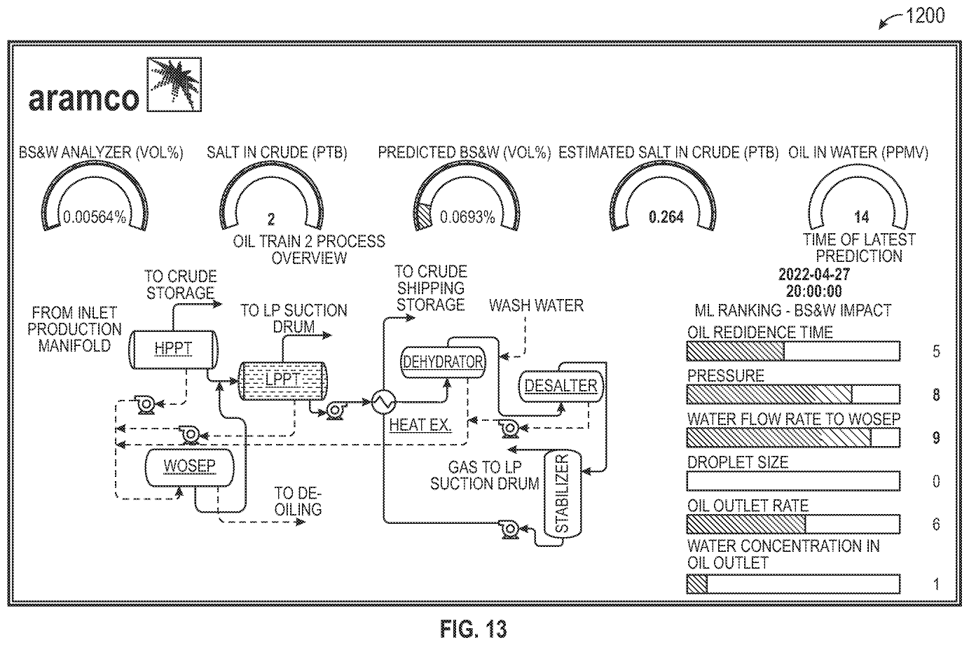

FIG. 13 shows the display 1200 of FIG. 12 with a BS&W in the oil exiting the GOSP at 0.00564 vol %. Since the BS&W is lower than the standard of 0.2 vol %, the ML model recommends not changing any of the process parameters and exiting the feedback loop.

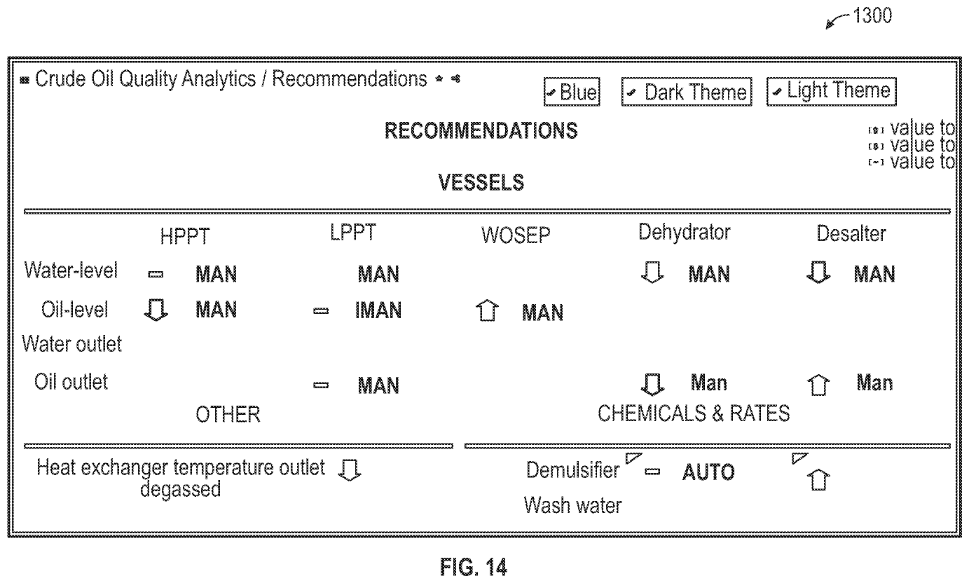

FIG. 14 shows recommendations of the ML model. The recommendations are shown on a screen 1300. The screen 1300 shows a grid with five columns and four rows. The five columns are HPPT, LPPT, WOSEP, Dehydrator, and Desalter. The four rows are water-level, oil-level, water outlet, and oil outlet. The recommendations of the ML model are shown by a dash, upward arrow, or downward arrow at a certain column and a certain row.

For example, a dash at the HPPT column and the water-level row indicates that the ML model recommends not changing the water-level in the HPPT. A downward arrow at the HPPT column and oil-level row indicates that the ML model recommends manually reducing the oil level in the HPPT.

Returning to a step 418 in FIG. 10, the operator changes the process parameters and enters the changed process parameters to the feedback loop and the feedback loop starts over as described in step 320 of FIG. 3.

In one or more embodiments, the operator uses Natural Language Processing (NLP) to maintain or reverse a change in the process parameters. NLP is a subfield of artificial intelligence in connection with the interactions between computers and human language. In particular, NLP is a computer program to process and analyze large amounts of data of natural language. A computer equipped with NLP understands the concentrations of the human language. NLP then accurately extracts information contained in the language. In one or more embodiments, the NLP comprises speech recognition, natural language understanding, and natural language generation.

In the NLP step, inputs from an operator is used to improve the ML model. The data from the operator is sent through the NLP tool. The output of the NLP tool is inputted to the ML model for further processing.

Next the changed process parameters are entered into the feedback loop (see step 410) replacing the total parameters and the feedback loop is repeated.

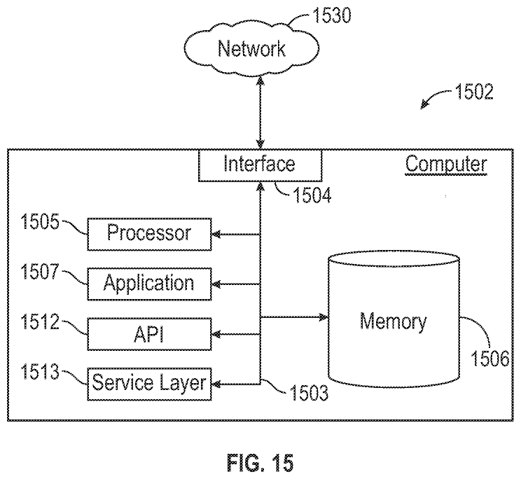

Embodiments may be implemented on a computer system. FIG. 15 is a block diagram of a computer system 1502 used to provide computational functionalities associated with described algorithms, methods, functions, processes, flows, and procedures as described in the instant disclosure, according to an implementation. The illustrated computer 1502 is intended to encompass any computing device such as a high performance computing (HPC) device, server, desktop computer, laptop/notebook computer, wireless data port, smart phone, personal data assistant (PDA), tablet computing device, one or more computer processors within these devices, or any other suitable processing device, including both physical or virtual instances (or both) of the computing device. Additionally, the computer 1502 may include a computer that includes an input device, such as a keypad, keyboard, touch screen, or other device that can accept user information, and an output device that conveys information associated with the operation of the computer 1502, including digital data, visual, or audio information (or a combination of information), or a GUI.

The computer 1502 can serve in a role as a client, network component, a server, a database or other persistency, or any other component (or a combination of roles) of a computer system for performing the subject matter described in the instant disclosure. The illustrated computer 1502 is communicably coupled with a network 1530. In some implementations, one or more components of the computer 1502 may be configured to operate within environments, including cloud-computing-based, local, global, or other environment (or a combination of environments).

At a high level, the computer 1502 is an electronic computing device operable to receive, transmit, process, store, or manage data and information associated with the described subject matter. According to some implementations, the computer 1502 may also include or be communicably coupled with an application server, e-mail server, web server, caching server, streaming data server, business intelligence (BI) server, or other server (or a combination of servers).

The computer 1502 can receive requests over network 1530 from a client application (for example, executing on another computer 1502) and responding to the received requests by processing the said requests in an appropriate software application. In addition, requests may also be sent to the computer 1502 from internal users (for example, from a command console or by other appropriate access method), external or third-parties, other automated applications, as well as any other appropriate entities, individuals, systems, or computers.

Each of the components of the computer 1502 can communicate using a system bus 1503. In some implementations, any or all of the components of the computer 1502, both hardware or software (or a combination of hardware and software), may interface with each other or the interface 1504 (or a combination of both) over the system bus 1503 using an application programming interface (API) 1512 or a service layer 1513 (or a combination of the API 1512 and service layer 1513. The API 1512 may include specifications for routines, data structures, and object classes. The API 1512 may be either computer-language independent or dependent and refer to a complete interface, a single function, or even a set of APIs. The service layer 1513 provides software services to the computer 1502 or other components (whether or not illustrated) that are communicably coupled to the computer 1502. The functionality of the computer 1502 may be accessible for all service consumers using this service layer. Software services, such as those provided by the service layer 1513, provide reusable, defined business functionalities through a defined interface. For example, the interface may be software written in JAVA, C++, or other suitable language providing data in extensible markup language (XML) format or other suitable format. While illustrated as an integrated component of the computer 1502, alternative implementations may illustrate the API 1512 or the service layer 1513 as stand-alone components in relation to other components of the computer 1502 or other components (whether or not illustrated) that are communicably coupled to the computer 1502. Moreover, any or all parts of the API 1512 or the service layer 1513 may be implemented as child or sub-modules of another software module, enterprise application, or hardware module without departing from the scope of this disclosure.

The computer 1502 includes an interface 1504. Although illustrated as a single interface 1504 in FIG. 15, two or more interfaces 1504 may be used according to particular needs, desires, or particular implementations of the computer 1502. The interface 1504 is used by the computer 1502 for communicating with other systems in a distributed environment that are connected to the network 1530. Generally, the interface 1504 includes logic encoded in software or hardware (or a combination of software and hardware) and operable to communicate with the network 1530. More specifically, the interface 1504 may include software supporting one or more communication protocols associated with communications such that the network 1530 or interface's hardware is operable to communicate physical signals within and outside of the illustrated computer 1502.

The computer 1502 includes at least one computer processor 1505. Although illustrated as a single processor 1505 in FIG. 15, two or more computer processors may be used according to particular needs, desires, or particular implementations of the computer 1502. Generally, the computer processor 1505 executes instructions and manipulates data to perform the operations of the computer 1502 and any algorithms, methods, functions, processes, flows, and procedures as described in the instant disclosure.

The computer 1502 also includes a memory 1506 that holds data for the computer 1502 or other components (or a combination of both) that can be connected to the network 1530. For example, memory 1506 can be a database storing data consistent with this disclosure. Although illustrated as a single memory 1506 in FIG. 15, two or more memories may be used according to particular needs, desires, or particular implementations of the computer 1502 and the described functionality. While memory 1506 is illustrated as an integral component of the computer 1502, in alternative implementations, memory 1506 can be external to the computer 1502.

The application 1507 is an algorithmic software engine providing functionality according to particular needs, desires, or particular implementations of the computer 1502, particularly with respect to functionality described in this disclosure. For example, application 1507 can serve as one or more components, modules, applications, etc. Further, although illustrated as a single application 1507, the application 1507 may be implemented as multiple applications 1507 on the computer 1502. In addition, although illustrated as integral to the computer 1502, in alternative implementations, the application 1507 can be external to the computer 1502.

There may be any number of computers 1502 associated with, or external to, a computer system containing computer 1502, each computer 1502 communicating over network 1530. Further, the term “client,” “user,” and other appropriate terminology may be used interchangeably as appropriate without departing from the scope of this disclosure. Moreover, this disclosure contemplates that many users may use one computer 1502, or that one user may use multiple computers 1502.

In some embodiments, the computer 1502 is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, cloud computing system may operate according to one or more service models, such as infrastructure as a service (IaaS), platform as a service (PaaS), software as a service (SaaS), mobile “backend” as a service (MBaaS), serverless computing, and/or function as a service (FaaS).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method for increasing the quality of crude oil exiting a gas-oil separation plant (GOSP), wherein the GOSP comprises sensors that determine process parameters of the crude oil, the method comprising the steps:
determining, from the process parameters, WiO-parameters that depend on the concentration of water in the crude oil (WiO);
determining virtual parameters of the crude oil;
determining total parameters by adding the virtual parameters to the WiO-parameters;
performing a feedback loop, comprising the steps:
changing one or more of the total parameters,
determining the quality of the crude oil exiting the GOSP, wherein
when the quality is improved, the change in the one or more total parameters is maintained,
when the quality is worsened, the change in the one or more total parameters is reversed; and
repeating the feedback-loop as long as the quality of the crude oil exiting the GOSP increases.

2. The method according to claim 1, wherein the GOSP comprises a high pressure production trap (HPPT), a low pressure production trap (LPPT), a dehydrator, a desalter, and a water oil separator (WOSEP).

3. The method according to claim 2, wherein the process parameter comprise a flow rate of crude oil from the HPPT to the LPPT, a flow rate of crude oil from the LPPT to the dehydrator, a flow rate of crude oil from the dehydrator to the desalter, a flow rate of crude oil from the desalter to the WOSEP.

4. The method according to claim 2, wherein the process parameters further comprise a salt concentration exiting the desalter, and concentration of water exiting the LPPT.

5. The method according to claim 1, wherein the crude oil comprises an emulsion that comprises oil in water (OiW) and water in oil (WiO), and the process parameter comprise the size of the droplets of the OiW and the WiO in the HPPT, LPPT, dehydrator, desalter, and WOSEP.

6. The method according to claim 1, wherein feedback loop is performed by a machine learning (ML) model.

7. The method according to claim 6, wherein the ML model uses a gradient boosting algorithm, scikit learn's gradient boosting regressor, or random forest regressor.

8. The method according to claim 1, wherein the quality of the crude oil exiting the GOSP comprises concentrations of WiO, basic sediment and water (BS&W), salt, and water in the crude oil exiting the GOSP.

9. The method according to claim 1, wherein the virtual parameters comprise slugging and surge in a pipeline of the GOSP.

10. The method according to claim 1, further comprising: aggregating redundant process parameters.

11. The method according to claim 1, further comprising
determining, from the process parameters, BS&W-parameters that depend on the BS&W of the crude oil exiting the GOSP,
determining total parameters by adding the BS&W-parameters to the WiO-parameters.

12. The method according to claim 1, further comprising
determining, from the process parameters, salt-parameters that depend on the salt concentration of the crude oil exiting the GOSP,
determining total parameters by adding the salt-parameters to the WiO-parameters.

13. The method according to claim 1, wherein the process parameters are preprocessed after the process parameters are determined by the sensors.

14. A non-transitory computer readable medium storing instructions executable by a computer processor, the instructions comprising functionality for:
determining, from the process parameters, WiO-parameters that depend on the concentration of water in the crude oil (WiO);
determining virtual parameters of the crude oil;
determining total parameters by adding the virtual parameters to the WiO-parameters;
performing a feedback loop, comprising the steps:
changing one or more of the total parameters,
determining the quality of the crude oil exiting the GOSP, wherein
when the quality is improved, the change in the one or more total parameters is maintained,
when the quality is worsened, the change in the one or more total parameters is reversed; and
repeating the feedback-loop as long as the quality of the crude oil exiting the GOSP increases.
